(19) United States
(12) Patent Application Publication
(10) Pub. No.: US 2026/0054059 A1
(43) Pub. Date: Feb. 26, 2026

(54) DYNAMIC MODULATION OF ELECTRICAL VARIABLES IN NERVE STIMULATION TO PRODUCE FUNCTIONAL MUSCLE MOVEMENT WITHOUT MUSCLE FATIGUE

(71) Applicant: Asayena, Inc., La Jolla, CA (US)

(72) Inventors: Mickey Ellis ABRAHAM, La Jolla, CA (US); Ronald SAHYOUNI, Mission Viejo, CA (US); Herbert Tsvi GOLDENBERG, La Jolla, CA (US)

(73) Assignee: Asayena, Inc., La Jolla, CA (US)

(21) Appl. No.: 19/287,181

(22) Filed: Jul. 31, 2025

Related U.S. Application Data
(63) Continuation of application No. PCT/US24/14294, filed on Feb. 2, 2024.
(60) Provisional application No. 63/443,046, filed on Feb. 3, 2023.

(51) Int. Cl. A61N 1/36; A61N 1/05

(52) U.S. Cl. CPC A61N 1/36003; A61N 1/0556; A61N 1/36132; A61N 1/36139; A61N 1/36157; A61N 1/36175

(57) ABSTRACT

Peripheral neuromodulation systems and methods are provided for activating peripheral nerves for functional motor control. The systems include a multichannel electrode configured to be placed on or around a peripheral nerve of the subject, a pulse generator configured to deliver a stimulation to the nerve via the multichannel electrode to induce a functional limb movement for the subject, where the stimulation includes a series of current pulses including at least one pulse delay, and a controller operatively coupled to the pulse generator.

### CROSS-REFERENCE

This application is a continuation of PCT Application No. PCT/US24/14294, filed Feb. 2, 2024, which claims the benefit of U.S. Provisional Application No. 63/443,046, filed Feb. 3, 2023, the content of each of which is incorporated herein by reference.

The subject matter of this application is related to the following copending patent application: International Application No. PCT/US22/39202 (Attorney Docket No. 61435-703.601) which claims the benefit of U.S. Provisional Patent Application No. 63/228,754, filed Aug. 3, 2021, the entire contents of which are incorporated herein by reference.

### BACKGROUND

The present disclosure is in the medical and biomedical field, and more specifically in the field of treating or reducing the impact of weakness or paralysis (including the mitigation of muscle atrophy and contracture formation, maintenance of muscle bulk, and modulation of spasticity) and peripheral neuromodulation.

Damage to movement-controlling areas of the brain, spinal cord, and central nervous system (CNS) may result in weakness and paralysis. Signals sent from the injured CNS are too compromised to communicate with the working peripheral nervous system (PNS). Without this working connection, the PNS cannot initiate movement or the efferent signals are too weak to generate full muscle strength. This results in a number of clinical consequences, including weakness and/or paralysis, as well as the loss of muscle bulk, muscle atrophy, formation of contractures, and/or spasticity. Additionally, in a peripheral nerve injury, signals cannot reach the end-organ given nerve severance or other damage, creating a functional break in circuitry. Stroke is one of the most common CNS injuries, leaving approximately 40 million post-stroke patients internationally with paralysis. Other etiologies of paralysis and pathologies of the central and peripheral nervous system that lead to weakness, include spinal cord injury (SCI), multiple sclerosis (MS), amyotrophic lateral sclerosis (ALS), traumatic brain injury (TBI), cerebral palsy (CP), and peripheral nerve injuries.

Functional improvement of these deficits is currently predicated on extensive rehabilitation, on an order of months to years, as well as complex surgeries related to joint fusion, tendon transfers, and nerve transfers which are invasive, morbid, and have additional significant recovery time. Prior studies demonstrate that gains plateau at six months for patients with weakness and those with paralysis often have no functional rehabilitative gains whatsoever. Patients are often left with permanent disability and may not have access to wearable exoskeletons to try to improve function and quality of life.

The diseases are not an exhaustive list but are merely examples of weakness or paralysis that may be treated by the systems and methods disclosed herein.

### SUMMARY

A peripheral neuromodulation system is provided for activating peripheral nerves for motor control with specific parameters configured to induce fine muscle contractions without muscle fatigue. Furthermore, this neuromodulation system can be utilized in an autonomous closed loop fashion to restore functional limb movement, or be utilized in an open loop fashion to mitigate muscle atrophy, maintain muscle bulk, modulate spasticity and mitigate contracture formation to facilitate endogenous limb function. The disclosed peripheral neuromodulation system patent is configured to address all forms of weakness or paralysis and the clinical consequences thereafter caused by injury to the CNS or PNS that require motor control.

Aspects of the present disclosure provide an exemplary neuromodulation system for providing functional limb movement. The system may include a multichannel electrode configured to be placed on or around a peripheral nerve of the subject, a pulse generator configured to deliver a stimulation to the nerve via the multichannel electrode to induce a functional limb movement for the subject, where the stimulation includes a series of current pulses including at least one pulse delay, and a controller operatively coupled to the pulse generator.

The system may include a sensing lead configured to detect a volitional input of a patient, a multichannel electrode configured to be placed around a peripheral nerve, and a pulse generator configured to deliver a stimulation to the nerve to induce a functional limb movement, where the stimulation includes a series of current pulses including a pulse delay. The multichannel electrode may be a cuff configured to be placed around the nerve.

The controller may be configured to one or more of apply or modulate the stimulation based on volitional input. The controller may modulate the at least one pulse delay based on the volitional input. The controller may be implantable.

The volitional input may be based on one or more of a subject's nerve or muscular activity. In an example, the one or more of the subject's nerve or muscular activity is from a nerve or muscle different from the nerve to which the stimulation is applied or muscle associated with the nerve.

The system may include a manual trigger operatively coupled to the controller to receive input from the subject or other user, where the controller is configured to one or more of apply or modulate the stimulation based on the received input. This trigger can be used to allow the device to operate in a closed loop manner, in which volitional patient input can actuate the device resulting in meaningful motor movement. Furthermore, a sensory feedback mechanism can be employed to provide realtime feedback to the controller to allow for fine tuned motor movement and coordination based on the end users limb position and intended motion.

The pulse generator may be implantable or external and may modulate the stimulation and/or the pulse delay based on the volitional input. At least two successive pulses of the series of current pulses may be separated by the at least one pulse delay. In an example, each pulse of the series of current pulses may be separated by at least one pulse delay. The at least one pulse delay may be between 5 and 75 mS and may have an average of 40 mS. The pulses of the series of current pulses of the stimulation may have a pulse width between 50-1000 uS. The pulses of the series of current pulses of the stimulation may have a pulse current between 50-2000 uA. The pulses of series of current pulses are charged balanced, biphasic, or both.

The sensing lead may be implantable or externally adhered to the patient with an adhesive. The sensing lead may be an electromyographic sensor, an electroencephalography sensor, an electroencephalographic sensor, an adhesive electrode, or a manual on/off driver. The sensing lead may be configured to detect volitional input of a patient and operatively coupled to the controller.

The volitional input may be based on a patient's brain or nerve activity and/or a patient's muscular activity. Alternatively or in combination, the stimulation may be triggered by an input received from a manual trigger such as a handheld remote control or key fob like control element. In an example, the volitional input or sensing lead may be in communication with the manual trigger. In a further example, the manual trigger may be an external magnet configured to align with an internal coil in the pulse generator to serve as a trigger. The device can also function autonomously independent of the trigger mechanism. An exemplary method would include mitigation of muscle atrophy following an acute CNS injury by implanting the system and providing programmed therapeutic stimulation to the nerve to facilitate endogenous motor recovery.

An exemplary method for inducing flexion in a target muscle of a patient may include receiving an input from the subject or other user, and delivering, upon receiving of the input, a charge balanced symmetric biphasic stimulation waveform to the target muscle, including a series of current pulses and at least one pulse delay, thereby inducing a functional limb movement. Receiving the input may include detecting a volitional input of the subject.

The method may further include detecting a second volitional input of a patient; and modulating the stimulation waveform based on the second volitional input.

The method may further include adjusting at least one of an amplitude, frequency, pulse width, and pulse delay of the stimulation based on the volitional input.

The method may further include adjusting at least one of an amplitude, frequency, pulse width, and pulse delay of the stimulation based on the input received from the manual trigger.

The input may be received from the subject or other user via a manual trigger.

The method may further include detecting a sign of muscle fatigue.

The method may further include altering the pulse delay between at least two pulses of the series of pulses.

An exemplary method for providing dynamic stimulation for functional limb movement may include initiating flexion stimulation of a nerve with a constant current and a series of pulses including at least one pulse delay, detecting a volitional input of a patient, altering the pulse delay between two pulses of the series of pulses based on the volitional input as the flexion stimulation is maintained, and delivering a relaxing stimulation configured to evoking a controlled relaxation.

An exemplary method may utilize current and field steering to ensure activating of specific nerve fascicles for physiologic motion. The method may further include detecting a second volitional input of a patient and modulating the stimulation waveform based on the second volitional input. The method may further include adjusting at least one of an amplitude or intensity, frequency, pulse width, and pulse delay based on the volitional input. The method may further include detecting a sign of muscle fatigue and altering the pulse delay between at least two pulses of the series of pulses.

An exemplary method for providing dynamic stimulation for functional limb movement may include initiating stimulation of a nerve with a constant current and a series of pulses including a pulse delay, maintaining a stimulation by altering the pulse delay between two pulses of the series of pulses, and delivering a relaxing stimulation configured to evoking a controlled relaxation.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a peripheral neuromodulation system constructed for providing functional limb movement is provided including an implantable pulse generator (IPG), an implantable sensing lead configured to detect a volitional input, and a multichannel electrode (MCE) configured to be placed around a peripheral nerve according to certain embodiments.

FIG. 2 illustrates a peripheral neuromodulation system including an implantable receiver stimulator coil (RSC), an external pulse generator (EPG), and an external coil to power the implantable RSC according to certain embodiments.

FIGS. 3A-3C illustrates an MCE having an 8-channel MCE including two parallel electrode rings according to certain embodiments.

FIG. 4A is an image of an MCE with electrode rings, each with four rectangular electrodes in a silicon enclosure 900 apart according to certain embodiments.

FIG. 4B is a picture of an intraoperative image of an implanted MCE according to certain embodiments.

FIG. 5 illustrates neuromodulation system including an electrode assembly and a pulse generator including an external component and an implantable component according to certain embodiments.

FIG. 6 is a rolled-out view of the electrode assembly of FIG. 5 showing an arrangement of individual electrode elements according to certain embodiments.

FIG. 7 is a block diagram showing the circuitry in the external and implantable components of the pulse generator of FIG. 5 according to certain embodiments.

FIG. 8 illustrates a charge balanced symmetric biphasic waveform including a pulse delay according to certain embodiments.

FIG. 9 illustrates a waveform transition from a first frequency to a secondary frequency once muscle contraction has been initiated to mitigate muscle fatigue according to certain embodiments.

FIG. 10 is a flow diagram illustrating a method for providing dynamic stimulation paradigm for functional limb movement according to certain embodiments.

FIG. 11 is a flow diagram illustrating a method for providing neuromodulation for functional limb movement according to certain embodiments.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

A peripheral neuromodulation system is provided for activating peripheral nerves for motor control with specific parameters configured to induce fine muscle contractions without muscle fatigue. The disclosed peripheral neuromodulation system patent is configured to address all forms of weakness or paralysis caused by injury to the CNS or PNS that require motor control.

Direct nerve stimulation to modulate muscle movement can require a balance between obtaining maximal muscle contraction, which may require higher frequencies, and mitigating fatigue, which may require lower frequencies.

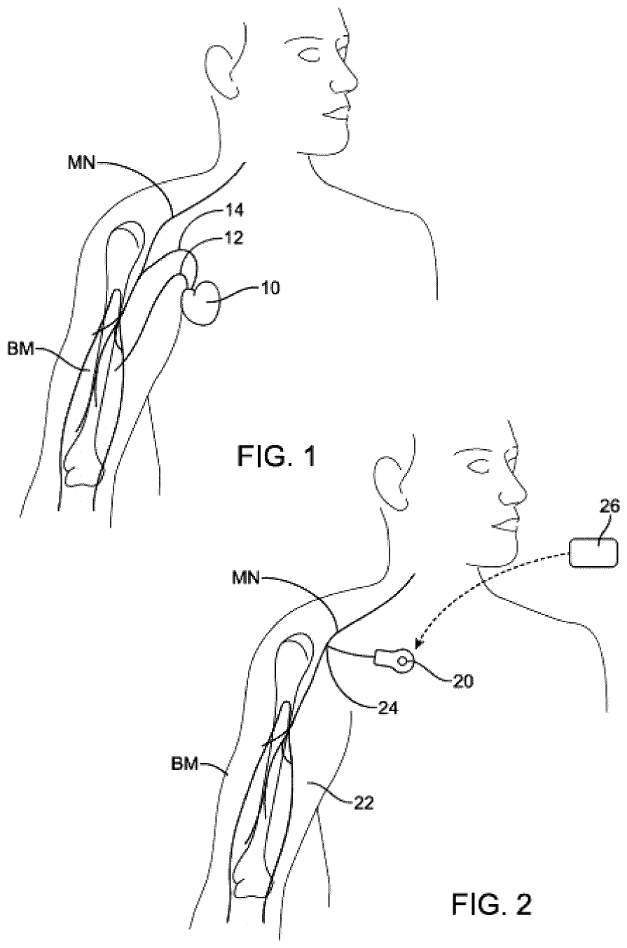

According to some embodiments, a neuromodulation system for providing functional limb movement is provided. Turning to FIG. 1, according to some embodiments, a neuromodulation system for providing functional limb movement is provided and which may include an implantable pulse generator (IPG) 10, an implantable sensing lead 12 configured to detect a volitional input, and a multichannel electrode (MCE) 14 configured to be placed around a peripheral nerve. The IPG may include electronics such as a controller and battery or other power source inside hermitic or titanium case. A surgeon may implant the IPG subcutaneously below the clavicle in the upper chest or in the axilla and connects the IPG to the sensing lead and a stimulation connected to the MCE.

According to some embodiments, the neuromodulation system can be configured in an autonomous mode using one or more sensing leads configured to detect one or more signals from a patient or volitional input such as electromyography (EMG), electroencephalography (EEG), and/or electroneurography (ENG). According to some embodiments, the volitional input may be based on a patient's nerve activity and/or a patient's muscular activity. According to some embodiments, the neuromodulation system is configured to detect nerve activity signals prior to an attempt of joint flexion. This may be achieved through detection of ENG signals, such that the specific electrode of the multichannel contact electrode that can sense the weakened ENG signal and serve as an input for the system, is relayed to that same equivalent contact on the distal multichannel contact electrode for the output. According to some embodiments, the neuromodulation system is configured to detect weak EMG signals when arm flexion is attempted and delivers stimulation current to one or more channels of the MCE 14 wrapped around a musculocutaneous nerve MN which innervates the patient's bicep muscles BM.

According to some embodiments, the EMG signal may be detected by the implantable sensing lead 12 or a surface electrode. In some embodiments, the signal is an EEG signal detected from an implantable or external EEG sensor. The sensing lead may be implantable or externally adhered to the patient with an adhesive. The sensing lead may be an electromyographic sensor, an electroencephalography sensor, an electroencephalographic sensor, or a manual on/off driver.

According to some embodiments, a neuromodulation system can be configured in an open loop with a manual trigger such as a remote control or key fob like control element. In some embodiments, the sensor is an on/off driver to activate the system. In an example, the manual trigger may be an external magnet configured to align with an internal coil in the pulse generator.

In an aspect, the MCE configured to encircle and selectively stimulate one or more motor nerves. In an aspect, placement of the MCE enables the graded electrical pulses generated by the pulse generator to be delivered in a controlled and localized manner ensuring the intended muscle groups are activated with precision. In an example, the MCE may include an 8-channel multichannel cuff electrode having two parallel electrode rings. In an example, each electrode ring may have four electrodes arranged 90 degrees apart. In an aspect, the electrodes may be made from typical nerve interfacing materials such as gold, platinum, platinum/iridium, or coated with a conducting polymer. In an example, the MCE 14 may have a 2-mm to 6-mm diameter and a 1-cm to 2-cm length cuff. The cuff electrode contacts may be arranged in two or more “rings” with reach ring containing four individual 2-mm×1-mm rectangular (tripolar) platinum or 90/10 platinum/iridium contacts embedded in silicone positioned at 0, 90, 180, and 270 degrees around the ring. Spacing from 1-mm to 5-mm is maintained between contacts with 1-mm space from contact to cuff edge. This arrangement can allow for monopolar stimulation of discrete neural locations as well as bipolar stimulation between two contacts. These dimensions are meant to be exemplary but are not meant to be limiting in any way.

In an example, the MCE 14 may have embedded sutures in the silicone to facilitate cuff placement. The surgeon can position the MCE around a patient's musculocutaneous nerve and connects the connector tip end of the lead to the IPG. The cuff electrodes can apply electrical current that stimulates the musculocutaneous nerve which causes the arm to flex at the elbow.

The electrode structure may distribute current over any number of electrode elements or other contacts, allowing for independent and/or synchronous activation of any number of electrodes. Each electrode element or contact may be configured as the anode or cathode during the active phase of the stimulus.

In an aspect, the pulse generator is configured to control a pulse intensity, pulse duration, and a pulse delay of the stimulation. In an aspect, the pulse generator is configured to deliver graded electrical pulses. In an aspect, the pulse generator is configured to deliver an electrical pulse to a target motor nerve based on the characteristic, such as amplitude, of the volitional input.

In an aspect, the pulse generator is configured to deliver electrical pulses in a graded fashion over a programmable amount of time by delivering pulses with increasing amplitude over time to allow for smooth muscle activation. By adjusting the intensity and duration of stimulation, the system can allow for fine-tuned control over the speed, strength, and extent of motor movement. For instance, a lower level of electrical stimulation over an extended ramp time would result in a gentle muscle movement, ideal for fine motor control. Conversely, a higher level of electrical stimulation over a shorter ramp time may lead to a stronger muscle contraction, suitable for more forceful movements.

In an aspect, the pulse generator includes a microprocessor, power source (e.g., battery), and control software. The pulse generator may also continuously monitor the sensors to detect muscle activity and identify changes in EMG signals when the patient intends to move. The detected EMG signal is transmitted to the microprocessor for real-time analysis. The microprocessor processes the EMG signal, specifically focusing on its amplitude, which correlates with the patient's intended muscle force. Based on this analysis, the microprocessor adjusts the parameters of the electrical pulse to be delivered including pulse amplitude (strength), pulse duration, and pulse frequency. The microprocessor may generate an electrical pulse tailored to the patient's intent, modulating the electrical pulse strength in response to the amplitude of the EMG signal. In an aspect, the pulse generator may be configured to deliver graded electrical pulses with increasing amplitudes over time to allow for smooth muscle activation. The graded electrical pulse may be subsequently sent to the multichannel electrode for precise delivery to the motor nerve.

Stimulation parameters may be programmed, such as intensity (range 0.1 mA to 2.5 mA), pulse width (range 10 μs to 500 μs), and frequency (range 10 Hz to 50 kHz). The stimulation waveform will typically be biphasic, asymmetric and charge balanced, with a delay of 100 μs between the active and recovery phases. Current intensity can range from 40 μA to 2000 μA.

According to some embodiments, the neuromodulation system may include one or more cuff electrodes which may be configured to stimulate one or more nerves and/or multiple locations on a single nerve. Each cuff electrode may contain one or more contacts used to stimulate the nerve. Electrode configurations may be as simple as a single ring electrode or may be more complex with a plurality rings.

According to some embodiments, the neuromodulation system may include a volitional electrode or sensor for providing information to the system to dynamically adjust stimulation parameters in real-time to execute a movement. According to some embodiments, the neuromodulation system may include an EMG sensor configured to detect volitional input signals generated by the patient's muscles, signifying their intent to initiate motor movements.

In an aspect, the EMG sensor is configured to continuously monitor the patient's muscle activity to detect their intention to initiate a motor movement. This input can be used as a trigger signal for the subsequent neuromodulation process.

According to some embodiments, the neuromodulation system may include an EMG adhesive or sticker electrode which may be placed on the skin over any functional muscle in the body to provide a non-invasive means for acquiring volitional input from any muscle in the body. In an aspect, the EMG sticker electrode provides an alternate or additional input source to the implanted EMG sensing lead. In an example, the EMG sensor may communicate with the EPG in a wired or wireless fashion. In an example, the volitional input may be delivered to the RSC or the IPG via short-range RF telemetry.

In an aspect, the EMG sensor may include surface electrodes or implanted electrodes, tailored to the specific application. Surface electrodes may be non-invasive and can adhere to the skin surface above the target muscles, while implanted electrodes are surgically positioned closer to or within the muscles for more direct access to muscle activity. Surface electrodes can capture electrical signals generated by muscle fibers during contraction, while implanted electrodes provide even more direct access to muscle activity.

In some embodiments, the neuromodulation system will include two cuff electrodes. One will act as the input driver by sensing weak ENG activity and will communicate to the IPG. The IPG may then send specific parameters to a second distal cuff electrode to activate the nerve fascicles and actuate motor control.

In some embodiments, the neuromodulation system will include an on/off control. This on/off control may communicate with the IPG to power the stimulation on or off.

According to some embodiments, the neuromodulation system may include wearable devices including accelerometers, gyroscopes, position sensors, and/or implanted devices able to detect EMG activity, motor unit recruitment, and other measures of limb location. This sensory feedback would provide realtime modulation of stimulation parameters to facilitate the completion of the intended motor movement of the end user.

The IPG can be configured to wirelessly interface with an external handheld device. The handheld device may be placed on the skin over the implant to provide a non-invasive means for the patient to activate the IPG, to adjust the stimulation parameters (within the physician prescribed limits), to check battery status, and to optionally charge wirelessly. The IPG will typically be MRI compatible and have the ability to be wirelessly charged via a transcutaneous magnetic charging coil.

The IPG can be further configured to wirelessly interface with a physician programmer which may comprise a tablet computer and a telemetry cable having a telemetry head. The telemetry head may communicate with the IPG through the skin via short-range radiofrequency (RF) telemetry. Telemetry communication can allow the physician to non-invasively interrogate and configure the IPG settings. In an example, the physician programmer has the capability to monitor EMG waveforms, configure stimulation modes, adjust stimulation parameter values, and store waveforms and settings.

Turning to FIG. 2, according to some embodiments, a neuromodulation system for providing functional limb movement is provided including an implantable receiver stimulator coil (RSC) 20 connected to an MCE 24 by a stimulation lead. The RSC is typically MR compatible. The surgeon may implant the RSC 22 subcutaneously below the clavicle in the upper chest in the axilla or in the arm and connects to the stimulation lead. The MCE 24 typically comprises a cuff having a 2-mm to 6-mm diameter and a 1-cm to 2-cm length. The cuff electrode contacts can be arranged in two or more “rings” with reach ring containing four individual 2-mm×1-mm rectangular (tripolar) platinum or 90/10 platinum/iridium contacts embedded in silicone positioned at 0, 90, 180, and 270 degrees around the ring. The contacts have a 1-mm to 5-mm spacing with a 1-mm space from contact to cuff edge. This arrangement can allow for monopolar stimulation of discrete neural locations as well as bipolar stimulation between two contacts. The cuff may optionally have embedded sutures in the silicone to facilitate cuff placement. The surgeon typically positions the MCE around a patient's musculocutaneous nerve and connects the connector tip end of the lead to the RSC. The cuff electrodes may be configured to apply electrical current that stimulates the musculocutaneous nerve to cause the arm to flex at the elbow.

In contrast to the first embodiment, the alternative system employees an external pulse generator (EPG) and external coil to power the implanted RSC. The EPG and coil are typically disposed in a housing 26 which may be located over the RSC 20, as shown by arrow 28 in FIG. 2, to align the external and internal coils. The EPG may contain electronics and battery inside titanium case and comprises or is connected to an external EMG sensor and further comprises a processor or controller which is programmed with an algorithm which detects weak electromyography signals from the EMG sensor when arm flexion is attempted. The algorithm may be further programmed to deliver because the EPG to deliver stimulation to one or more channels of the MCE wrapped around the musculocutaneous nerve. In an example, the volitional input from the sticker electrode will be sent to the EPG, which will typically be magnetically attached externally and interface through the skin with the implanted RSC.

The alternative systems will typically further comprise a movement remote, typically a hand-held device configured to be placed on the skin over the implant and to provide a non-invasive means for the patient to activate the RSC, to adjust the stimulation parameters (within the physician prescribed limits), and to check battery status.

The alternative systems will also typically comprise a physician programmer similar to that of the first embodiment, for example comprising a tablet computer and a telemetry cable/head which communicates with the IPG through the skin via short-range radiofrequency (RF) telemetry, allowing the physician to noninvasively interrogate and configure the IPG settings. The physician programmer can have the capability to monitor EMG waveforms, configure stimulation modes, adjust stimulation parameter values, and store waveforms and settings.

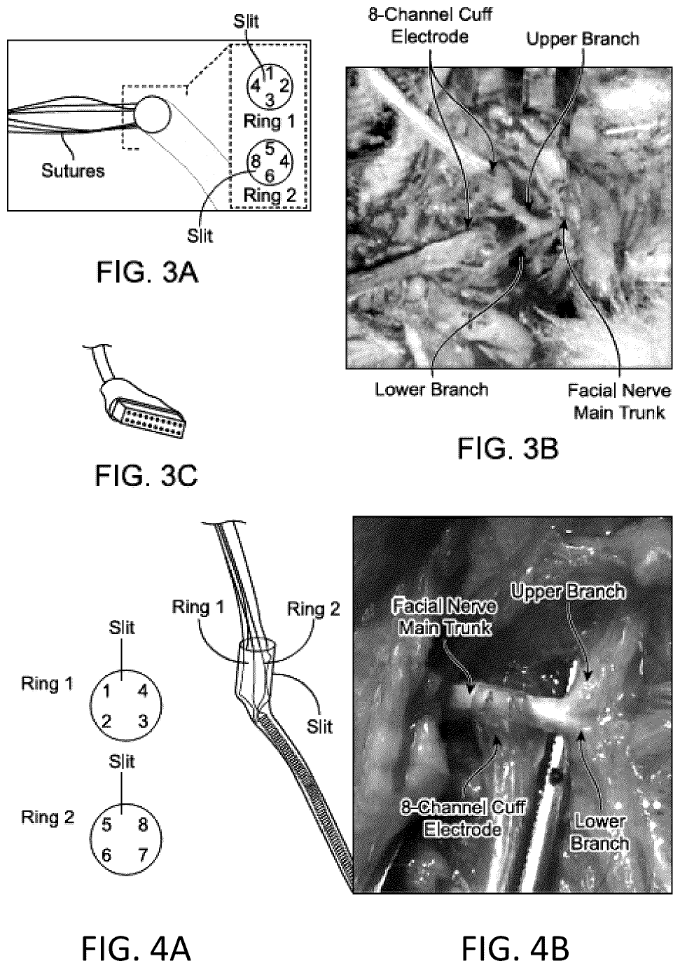

Turning to FIGS. 3A-3C and 4A-4B, an implanted multichannel electrode is shown. In an example, a multichannel electrode may be a cuff electrode as sold by MicroProbes for Life Science (Gaithersburg, MD, USA). Each cuff had an inner diameter of 1.5 mm, with two separate rings of four, 100 micrometer, rectangular, electrode contacts arranged concentrically every 90 degrees (0°, 90°, 180°, 270°) within a silicon enclosure. The arrangement of the rings and contacts allows for monopolar stimulation of unique spatial locations on the nerve. The second parallel ring enables field steering, in which two electrodes of the same cuff may be stimulated simultaneously to elicit an amplified response. The charge injection capacity was −164 μC/cm2 at 1 mA with a phase duration of 82 uS (0.5-1.5 uC/phase). The electrode impedance was 0.5 kΩ at 1000 Hz.

In an example, the MCE may have an 8-channel MCE including two parallel electrode rings, each having four rectangular electrodes arranged 90 degrees apart within a silicone enclosure. FIG. 3B shows an intra-operative image of the two MCEs implanted at the upper and lower FN branches of Cat. FIG. 3C is an image of a male Omnetics connector originating from the MCE.

In an example, the neuromodulation system may include operational stimulation parameters that are monophasic or biphasic, with a current typically in a range from 0.1 to 20 mA, at a repetition in a range from 1 to 50 pulses per sec and duration in a range from 10 to 200 μs. The device has been designed and built to have a physician-programmable and be patient-adaptable via machine-learning capabilities to mimic normal use of arm flexion, for example.

Turning to FIG. 4A, an MCE with 2 electrode rings, each with 4 rectangular (1.5×0.25×0.038 mm) platinum electrodes in a silicon enclosure 900 apart according to certain embodiments. In an example, the current source may be controlled by an 8-channel digital-to-analog converter (TDT RX8) and configured to deliver biphasic electrical 82 μs pulses according to certain embodiments.

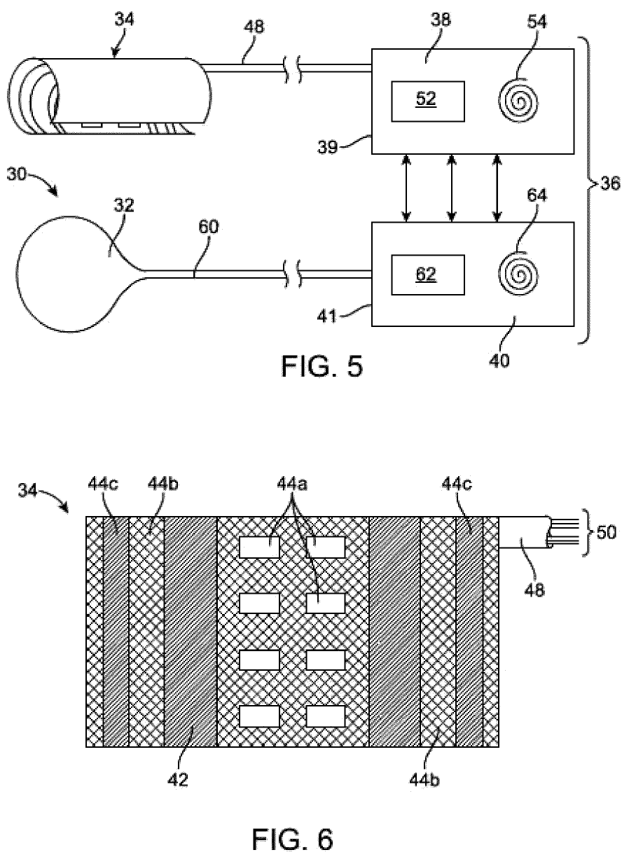

Turning to FIG. 5, a system 30 for actuating a target muscle in accordance with the principles of the present invention will be described. The system 30 may include an EMG sensor 32, an electrode assembly 34, and a pulse generator 36. The pulse generator 36 may include an implantable component 38 and an external component 40, where the external component is connected to the electrode assembly 34 by a lead 48 and the implantable component 40 is connected to the EMG sensor 32 by a lead 60. The implantable component 38 may include circuitry 52 and an inductive coil 54 located within an implantable housing 39, and the external component 40 may include circuitry 62 and an inductive coil 64 located within an external housing 41. The implantable housing 39 can be adapted or configured to be implanted subcutaneously in any of the locations described above, such as below the clavicle in the upper chest or in the axilla, and the external housing 41 is adapted or configured to be secured to patient's skin at a location proximate the location of the implanted housing 39, preferably directly overlying the implanted housing to enhance communication between the external and implanted components of the pulse generator 36. Optimally, magnetic coupling elements (schematically indicated by broken lines 46 in FIG. 7) may be provided to help locate and immobilize the external housing 41 over the implanted housing 39.

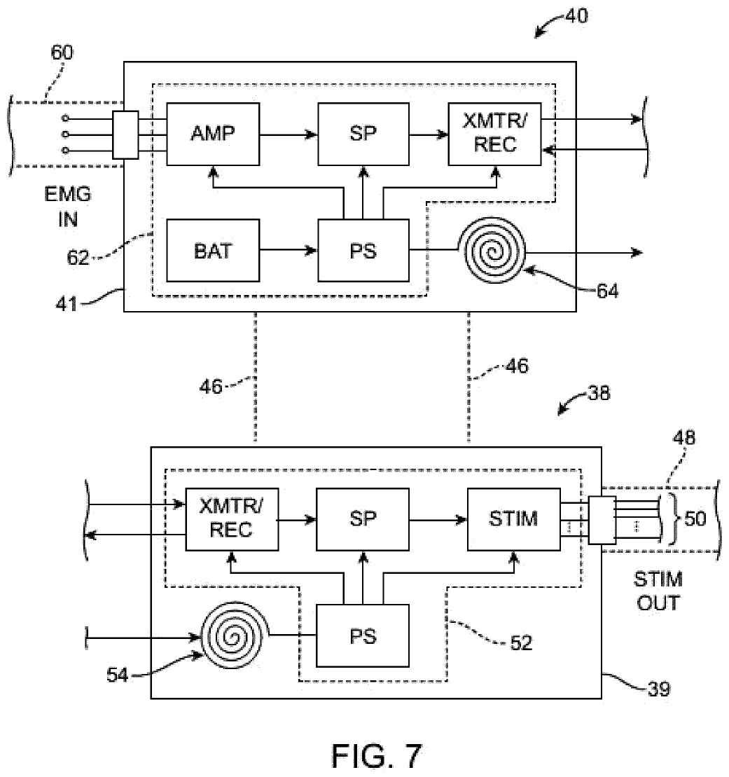

Turning to FIG. 6, the electrode assembly 34 may comprise a cuff 42, typically a multi-electrode cuff having a plurality of electrode elements 44 formed over an inner surface thereof. The electrode elements 44 can be formed on an inner surface of a backing or other support matrix for the cuff 42, and the backing may be folded or rolled over a target nerve to engage some or all the electrode elements against an outer surface of the nerve. The electrode elements may comprise different sizes and orientations where, for example, two, three, four or more channel electrodes 44a maybe circumferentially distributed over the inner surface of the backing so that they circumscribe the nerve when the backing is rolled or folded over the nerve. Additionally or alternatively, ring electrodes 44b may be formed to continuously circumscribe the inner surface of the cuff, and ground electrodes 44c may be provided when bipolar operation is desired. Alternatively, of course, any two of the channel electrodes 44 or one or more channel electrodes and a ring electrode and/or ground may be connected to operate in a bipolar mode.

Referring now to FIG. 7, further description of the circuitry 52 and 62 in the implantable component 38 and external component 40, respectively, will be provided. The circuitry 52 in the implantable component 38 typically may include a transmitter/receiver XMTR/REC configured for transcutaneous transmission and reception of low power data (digital and/or analog) between the implantable component 38 and the external component 40. The transmitter/receiver can receive power from a power supply PS which also provides power to a signal processing unit SP and a stimulator unit STIM. The power supply PS typically may include a battery or capacitor which may be recharged via inductive coil 54 which receives charge from the inductive coil 64 in the external component 40. The signal processing unit SP may be programmed to receive instructions from the external component 40 and to control and/or adjust parameters of the stimulator STIM in accordance with those instructions, The stimulator STIM generates and selectively may deliver current to the individual wires or channels 50 of the lead 48 which is connected to the cuff 34 to actuate the target muscle.

The circuitry 62 in the external component 40 typically may include a transmitter/receiver XMTR/REC configured for transcutaneous transmission and reception of low power data (digital and/or analog) with the implantable component 38. The transmitter/receiver receives power from a power supply PS which also provides power to a signal processing unit SP and an amplifier AMP. The power supply PS in turn may be powered by a rechargeable battery that may be recharged in a wired or wireless manner as is common for handheld digital devices. Although no shown, the external component 40 will typically have a display and an I/O capability to enable programming and updating the internal logic. The amplifier AMP may be configured to be externally connected to the EMG sensor 12 by lead 60, although wireless communication may also be used.

According to some embodiments, a method for providing neuromodulation for functional limb movement is provided including a pulse delay or stimulation frequency configured to evoke a sustained muscle contraction without noticeable fatigue. The method may include stimulation of a peripheral motor nerve such as the sciatic and/or femoral nerve. According to some embodiments, the sensing lead may be placed in the biceps brachii muscle and configured to detect weak EMG signals during attempted arm flexion.

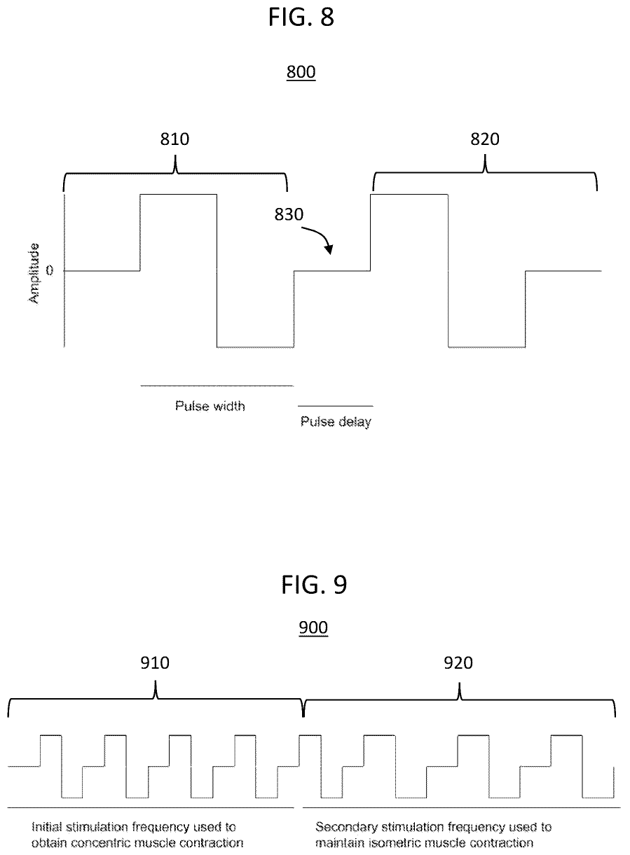

According to some embodiments, a method for providing neuromodulation for functional limb movement is provided including an interluded waveform 800. (See FIG. 8). According to some embodiments, the interluded waveform 800 may include a charge balanced symmetric biphasic stimulation waveform including a first pulse 810, a second pulse 820, and a pulse delay 830.

In an aspect, a critical factor for inducing functional stimulation without muscle fatigue is introducing a pulse delay tailored to the patient. The peripheral neuromodulation system may be configured to tailor stimulation parameters including the pulse delay from 10 mS to 70 mS to achieve this result.

According to some embodiments, the pulse delay may be any one of 5-10 mS, 10-15 mS, 15-20 mS, 25-30 mS, 35-40 mS, 45-50 mS, 55-60 mS, 65-70 mS, and 70-75 mS. In an example, the pulse delay modulation may have an average of 40 mS to elicit sustained isometric concentric contraction with no fatigue. In an aspect, the pulse delay modulation may be a time devoid of stimulation between at least two (2) adjacent pulses.

According to some embodiments, a method for providing neuromodulation for functional limb movement is provided including delivering a series of two or more interluded waveforms 900. (See FIG. 9). According to some embodiments, the interluded waveform 900 may include a waveform transition from a first frequency 910 configured to evoke a first muscle contraction to a second frequency 920 configured to evoke a second muscle contraction once muscle contraction has been initiated to mitigate muscle fatigue. By varying the pulse delay, current amplitude, and pulse width, a secondary set of stimulation parameters may be instantaneously transitioned to continue to stimulate the nerve and allow for muscle contraction but at a different stimulation parameter. We have found experimentally that increased pulse delays allow for less muscle fatigue which is useful for sustained muscle contraction as opposed to shorter pulse delays which are effective in inducing initial muscle contraction.

The method may include stimulation of a peripheral motor nerve both before and after an ischemic injury to the central nervous system (hemispheric stroke). By modulating the pulse delay, the stimulation may be instantaneous, graded, and controllable to allow for functional limb movement without nerve fatigue.

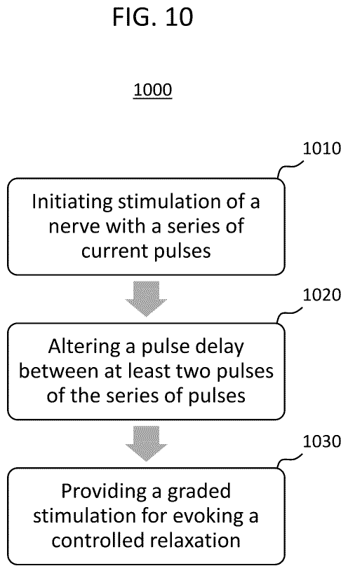

According to some embodiments, a method 1000 for providing dynamic stimulation paradigm for functional limb movement is provided including initiating stimulation of a nerve with a series of current pulses 1010, altering a pulse delay between at least two pulses of the series of pulses 1020, and providing a graded stimulation for evoking a controlled relaxation 1030. (See FIG. 10). In an example, controlled relaxation of a limb may be achieved when stimulating the femoral nerve at 200 uA or 100 uA, with a pulse width of 150 and 75 uS, respectively, and a pulse delay of 10 mS. In an aspect, each step of initiating, maintaining, and relaxing may be optimized to achieve the desired movement while mitigating off-target effects.

The optimization may be dynamically controlled by the patient, provider, or with the use of sensors providing information to the system to dynamically adjust stimulation parameters in real-time to execute a movement. In an aspect, movement may be sensed and modulated in real-time to allow for functional limb movement regardless of starting arm position or load.

According to some embodiments, a system is provided to dynamically modulate the stimulation parameters to actuate functional limb movements based on volitional control and subject intent. Experimentally, we found that when stimulating a nerve with constant current, altering the delay between pulses resulted in functionally relevant movement in the limb. For instance, a shorter pulse delay results in fast tetanic muscle activation but is limited by brisk muscle fatigue, while longer pulse delays result in slower muscle activation but less fatigue. There is an intermediary pulse delay that allows for strong muscle activation while mitigating muscle fatigue, this would be clinically relevant in patients and was seen experimentally in our porcine animal experiments. This optimal pulse delay allowed the muscle to remain in activated stimulation form for >5 minutes without fatigue.

For instance, stimulating at a high frequency (i.e., with a lower pulse delay) results in brisk concentric muscle contraction with fatiguability of the muscle over time. In an example, a high frequency may be between 60-100 Hz, while a lower frequency may be 10-40 Hz. Stimulation frequency may be correlated with pulse delay because the longer the pulse delay the lower the stimulation frequency. The muscle fatigue may cause the limb to relax against the force of gravity, which may not be functionally useful when attempting to generate isotonic concentric muscle movement to bring the hand up to the face, and then maintain this posture (i.e., isometric contraction).

Experimentally, we have elicited a spectrum of muscle contraction ranging in both strength and speed, with slow, medium, and fast limb movement with increasing current while maintaining the same frequency. Stimulation parameter frequency and amplitude spanned from 1-100 Hz and 0.01-0.20 mA, respectively. Based on the established Medical Research Council Manual Muscle Testing scale or manual muscle testing gradings system ranked 0-5, we were able to reliably and repeatedly elicit graded movements from 1-5. In an aspect, stable and repeatable movement was shown such that when a movement is elicited and graded response is achieved, the limb remains in its desired graded location for a specific amount of time without fatigue, including up to 5 minutes of constant stimulation, without any tissue damage on histology tests. Experimentally, we were able to maintain strong contraction without fatigue for at least five (5) minutes.

In a clinical setting, a therapy device that seeks to reanimate a limb to restore function will require the ability to generate sustained muscle contraction with no fatigue. In an aspect, an optimum stimulation setting may vary between nerves and over time when stimulating the same nerve. As the nerve recovers or the patient recovers over time, the stimulation parameters may have to be modulated to account for changes in the patients endogenous physiology. For example, by altering the pulse delay, or in this case increasing the pulse delay, isometric muscle contraction with no fatigue may be obtained for a sustained period. If the pulse delay is too long, however, this results in twitching movements of the limb that vary in frequency based on the pulse delay. Every patient will need to be individually programmed to establish their optimal pulse parameters that allows for smooth muscle contraction with minimal muscle fatigue.

Furthermore, the system can be programmed to delivery electrical stimulation to the nerve for purposes of maintaining muscle bulk and preventing muscle atrophy, as well as mitigating the formation of contractures as well as modulating spasticity. This configuration would facilitate endogenous patient recovery and serve more so as an augmentation to rehabilitation rather than an explicit therapeutic tool for limb reanimation.

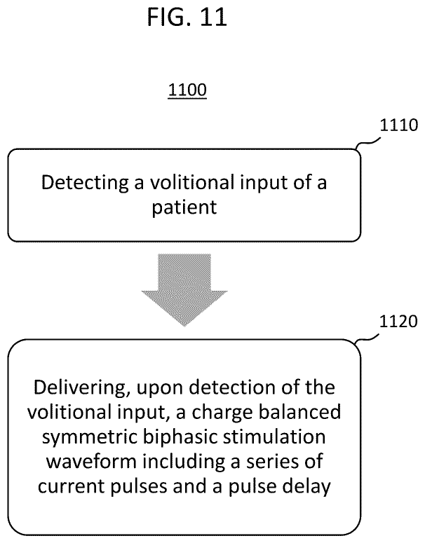

According to some embodiments, a method 1100 for providing neuromodulation for functional limb movement according to certain embodiments. The method may include a step 1110 of detecting a volitional input of a patient and a step 1120 of delivering, upon detection of the volitional input, a charge balanced symmetric biphasic stimulation waveform including a series of current pulses and a pulse delay. (See FIG. 11).

According to some embodiments, a method for providing neuromodulation for smooth limb movement is provided including a staged stimulation having a concentric isotonic stage, an isometric stage, and an isotonic eccentric stage. In an example, a smooth limb movement may include lifting a patient's hand up to their face, holding their hand to complete a task, and smoothly bringing the hand back down. In an example, the staged stimulation may include a series of stimulation paradigms that are executed to accomplish the smooth limb movement. If sensory components are integrated into the device allowing for detection of initial limb position, for example, these sensory signals would modulate the stimulation output of the device to account for variations in the subject's initial limb position while simultaneously facilitating the completion of a useful functional limb movement as intended by the end user.

In a concentric contraction, a muscle tension increases to meet a resistance and then remains stable as the muscle shortens. In an example, the concentric isotonic stage may include a stimulation algorithm configured to ramp up current delivery over a period of several seconds to smoothly contract a muscle to bring the limb to the desired height. Once the limb reaches the desired height, a maintenance stimulation at that height may be applied to prevent fatigue. In an aspect, by ramping up the stimulation in a graded fashion over a pre-determined period, the limb movement may be smooth and functional. In an aspect, the concentric isotonic stage is configured to prevent abrupt or erratic muscle contraction and subsequent limb movement as experienced in instantaneous current delivery. In an example, a concentric isotonic stage may include a first frequency 910 configured to evoke a first muscle contraction as shown in FIG. 9.

In an example, the isometric stage may include a stimulation algorithm that utilizes a different pulse delay and lower frequency then the initial stimulation parameter configured to maintain a smooth muscle contraction while preventing fatigue, tremor, or twitching. In an example, a fatigue is induced by a low pulse delay resulting with high stimulation frequencies on the order of 40-150 Hz. In an example, a tremor or twitching may be induced by a high pulse delay that results in high stimulation frequencies of 40-150 Hz. According to some embodiments, the isometric stage may be maintained in a closed or open loop manner. In an example, an open loop system may trigger based on a manual input to adjust the pulse delay to mitigate the undesired effects. In an example, a closed loop system may measure fatigue and/or tremor/twitching and dynamically adjust the pulse delay to mitigate the undesired effects.

According to some embodiments, the pulse delay may be tailored to that specific patient to optimize isometric movement and mitigate fatigue and debilitating twitching/tremor. The specific timing between pulses must be customized to the patient and varies based on several factors. These factors include the patients endogenous physiologic muscle activation, unique neuromuscular sensitivity to stimulation, and other factors including muscle bulk, contractures, and/or spasticity. One mechanism by which the optimum stimulation setting may be identified is clinically by modulating pulse delay manually and visualizing or measuring its effects on limb movement.

In an eccentric contraction, a muscle lengthens as the resistance becomes greater than the force the muscle is producing. An example would be lifting a heavy object that requires maximal stimulation of the nerve and subsequent muscle contraction to maximize limb movement.

Experimental results before and after an ischemic injury of the sciatic nerve and femoral nerve in a swine model demonstrated controlled relaxation of a limb. Prior to the ischemic injury, stimulation of the sciatic nerve with a 2-channel cuff with parameters of Current: 200 uA; Pulse Width: 100 uS; Pulse Delay 10 mS allows for instantaneous concentric muscle movement. Instantaneous concentric muscle movement was replicated with a 2-channel cuff around the femoral nerve at stimulation parameters of Current: 500 uA; Pulse Width: 50 uS; Pulse Delay 10 mS. Controlled relaxation of a limb was also achieved when stimulating the femoral nerve at 200 uA or 100 uA, with a pulse width of 150 and 75 uS, respectively, and a pulse delay of 10 mS. This controlled relaxation may be result of muscle fatigue but may also be modulated by decreasing the current over time to allow for a ramp-down in current with subsequent eccentric muscle relaxation.

By altering the stimulation parameters, the femoral nerve may elicit sustained isometric concentric contraction with no fatigue when stimulating at Current: 1500 uA; Pulse Width: 500 uS; Pulse Delay 40 mS.

Following the ischemic injury, the sciatic nerve was stimulated of with an 8-channel MCE which allowed for sustained contraction with no fatigue, by using the following parameters Current: 1500 uA; Pulse Width: 500 uS; Pulse Delay 40 mS. Isolated twitching movement of the sciatic nerve using an 8-channel cuff electrode following stroke may be elicited by modulating the pulse delay from 100 to 250 to 300 mS and using a constant current of 1500 uA and pulse width of 500 uS.

Muscle contraction may be sustained and provide force against resistance following an ischemic injury at 20, 60, 120, and 180 minutes post-ischemic injury by stimulating the sciatic nerve with an 8-channel cuff electrode using the following parameters: Current: 250 uA; Pulse Width: 250 uS; Pulse Delay 10 mS.

Instantaneous concentric limb movement was reliably observed at 20, 60, 120, and 180 minutes post-stroke in the femoral and sciatic nerves using both 2- and 8-channel electrodes, respectively, with stimulation parameters ranging from Current: 200 uA; Pulse Width: 50 uS; Pulse Delay 20 mS to Current: 2000 uA; Pulse Width: 200 uS; Pulse Delay 20 mS. Additional stimulation ranges that patients may benefit from are described below.

| | Broad range | Specific range | Exemplary range |
|---|---|---|---|
| Concentric muscle | Current: 50-2000 uA | Current: 50-500 uA | Current: 100-250 uA |
| contraction | Pulse Width: 50-1000 uS | Pulse Width: 50-500 uS | Pulse Width: 50-250 uS |
| (initial) | Pulse Delay: 5-500 mS | Pulse Delay: 5-75 mS | Pulse Delay: 5-50 mS |
| Isometric muscle | Current: 50-2000 uA | Current: 50-500 uA | Current: 100-250 uA |
| contraction | Pulse Width: 50-1000 uS | Pulse Width: 50-500 uS | Pulse Width: 50-250 uS |
| (maintenance) | Pulse Delay: 5-500 mS | Pulse Delay: 5-75 mS | Pulse Delay: 20-60 mS |
| Muscle | Current: 50-2000 uA | Current: 50-500 uA | Current: 100-250 uA |
| relaxation | Pulse Width: 50-1000 uS | Pulse Width: 50-500 uS | Pulse Width: 50-250 uS |
| | Pulse Delay: 5-500 mS | Pulse Delay: 5-75 mS | Pulse Delay: 20-60 mS |

For motor control, we discovered that pulse delay is the critical factor for achieving motor control without fatigue to induce a sustained contraction and movement. By varying the pulse delay, current amplitude, and pulse width, a secondary set of stimulation parameters may be instantaneously transitioned to continue to stimulate the nerve and allow for muscle contraction but at a different stimulation parameter. We have found experimentally that increased pulse delays allow for less muscle fatigue which is useful for sustained muscle contraction as opposed to shorter pulse delays which are effective in inducing initial muscle contraction.

According to some embodiments, the neuromodulation system may be configured to deliver a waveform with various stimulation parameters including waveforms at specific frequencies, ranging from 1-100 Hz. A typical waveform may range from 0-1000 μs cathodic phase duration, 0-1000 μs interphase interval and 0-1000 μs anodic phase duration, with a current of 0.01-20 mA.

According to some embodiments, the neuromodulation system may be configured as a closed loop system using an input trigger to detect volitional movement and to deliver stimulation or electrical current to a nerve based on the volitional movement to induce augmented muscle activation. In an example, the augmented muscle activation may be measured and/or monitored to dynamically adjust the stimulation parameters and control the functional muscle movement in real time. In an example, the input trigger may be detection of volitional EMG movement, electroencephalography (EEG) signals, electroneurogram (ENG) signals or another type of input providing volitional movement. In an example, EEG sensors may be used to detect when a patient thinks about moving their limb and provide the input trigger indicative of their brain activity. The input trigger can form as a patient signature induce muscle activation. The input trigger can be processed to trigger delivery of electrical current by the pulse generator to the cuff electrode. In an aspect, this patient signature may be programmed post-operatively such as in clinic to allow for useful limb movement.

In an example, the patient signature may be programmed to actuate a limb to move in a controlled fashion so as not to result in abrupt movement. In an aspect, the patient signature can be programmed to automatically adjust for a patient carrying objects of different weights/loads. In an aspect, when the patient signature for a movement previously programmed was too slow or fast, a real-time sensory monitoring of the speed of the limb movement may be used to regulate the current delivery to the nerve.

The input trigger can be provided from an implantable or wearable device. In an example, the neuromodulation system may include a wearable device configured to measure a volitional signal, limb speed, or other muscle parameters. In an example, the neuromodulation system may include an implanted device for measuring a volitional signal like EMG activation.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1.-49. (canceled)

50. A neuromodulation system for providing functional limb movement for a subject, the system comprising:
a multichannel electrode configured to be placed on or around a peripheral nerve of the subject;
a pulse generator configured to deliver a stimulation to the nerve via the multichannel electrode to induce a functional limb movement for the subject, wherein the stimulation includes a series of current pulses including at least one pulse delay; and
a controller operatively coupled to the pulse generator.

51. The system of claim 50, wherein the controller is configured to one or more of apply or modulate the stimulation based on volitional input.

52. The system of claim 51, further comprising a sensing lead configured to detect the volitional input of a patient and operatively coupled to the controller.

53. The system of claim 51, wherein the controller modulates the at least one pulse delay based on the volitional input.

54. The system of claim 51, wherein the volitional input is based on one or more of a subject's nerve or muscular activity.

55. The system of claim 52, wherein the one or more of the subject's nerve or muscular activity is from a nerve or muscle different from the nerve to which the stimulation is applied or muscle associated with the nerve.

56. The system of claim 50, further comprising a manual trigger operatively coupled to the controller to receive input from the subject or other user, wherein the controller is configured to one or more of apply or modulate the stimulation based on the received input.

57. The system of claim 50, wherein at least two successive pulses of the series of current pulses is separated by the at least one pulse delay.

58. The system of claim 50, wherein the at least one pulse delay is between 5 and 75 mS.

59. The system of claim 50, wherein the at least one pulse delay has an average of 40 mS.

60. The system of claim 50, wherein the pulses of the series of current pulses of the stimulation have a pulse width between 50-1000 uS.

61. The system of claim 50, wherein the pulses of the series of current pulses of the stimulation have a pulse current between 50-2000 uA.

62. The system of claim 50, wherein the pulses of series of current pulses are charged balanced, biphasic, or both.

63. The system of claim 50, wherein the pulse generator is implantable.

64. The system of claim 50, wherein the sensing lead is implantable.

65. The system of claim 50, wherein the controller is implantable.

66. The system of claim 50, wherein the sensing lead is an adhesive electrode.

67. The system of claim 50, wherein the sensing lead is an electromyographic sensor.

68. The system of claim 50, wherein the sensing lead is an electroencephalography sensor.

69. The system of claim 50, wherein the multichannel electrode is a cuff configured to be placed around the nerve.
